(19) United States
(12) Patent Application Publication
(10) Pub. No.: US 2026/0174463 A1
(43) Pub. Date: Jun. 25, 2026

(54) Catheter Assemblies for Thrombectomy or Atherectomy

(71) Applicant: Straub Medical AG, Vilters-Wangs (CH)

(72) Inventors: Tom Lavrijsen, Vilters-Wangs (CH); Mohsen Zendehbad, Vilters-Wangs (CH)

(21) Appl. No.: 19/129,636

(22) PCT Filed: Nov. 16, 2022
(86) PCT No.: PCT/EP2022/082092

§ 371 Date: May 13, 2025

(51) Int. Cl. A61B 17/3207; A61B 17/3203; A61M 25/00; A61M 25/01

(52) U.S. Cl. CPC A61B 17/320758; A61B 17/32037; A61M 25/0045; A61M 25/0068; A61M 25/0136

(57) ABSTRACT

A Catheter assembly for thrombectomy or atherectomy procedures, wherein the catheter assembly comprises: a catheter having a tubing comprising a tubular lumen extending in a length direction, and a distal end, a rotatable helix at least partially received in the tubular lumen and rotatable relative to the tubing, at least one sensor comprised by the catheter for detecting a condition during the procedure, wherein the condition is indicative of flow of bodily fluid and/or tissue through the tubular lumen and/or along the helix.

### TECHNICAL FIELD

The present disclosure relates to catheter assemblies for thrombectomy or atherectomy procedures, control units for controlling a catheter assembly in thrombectomy or atherectomy procedures, and handles for thrombectomy or atherectomy procedures.

### BACKGROUND

Flow of bodily tissue is important in aspiration procedures, such as in thrombectomy or atherectomy procedures. Detection thereof may occur by way of a sensor, such that a user needs to actively monitor the assembly for whether there is flow and, hence, aspiration, through the catheter, by checking the exhaust tube of the catheter receiving the removed bodily fluid and tissue, for example. Such “manual” check is prone to mistakes and depends on the individual practitioner. Accordingly, a need exists for catheter assemblies, control units, and systems that provide operational control to avoid undesirable flow and aspiration conditions.

### SUMMARY

The present disclosure is generally directed to improving operational control during atherectomy or thrombectomy procedures to avoid instances of reduced flow through the catheter.

According to one a first aspect, a catheter assembly includes a catheter having a tubing comprising a tubular lumen extending in a length direction, wherein the catheter comprises a distal end. In particular, the catheter's distal end may be distal to the catheter's tubing. The catheter assembly further comprises a rotatable helix at least partially received in the tubular lumen and rotatable relative to the tubing. The catheter assembly (optionally the catheter) comprises at least one (e.g. temperature, pressure and/or flow rate) sensor for detecting/monitoring/determining/measuring a condition during the procedure. This condition is indicative of flow of bodily fluid and/or tissue through the tubular lumen and/or along the helix.

The condition as detected/measured allows for a conclusion on the flow through the tubular lumen and/or along the helix. It is not necessary to measure the condition in the tubular lumen and/or along the helix (although this is conceivable). For example, the measurement may be taken from a position outside the catheter, e.g. distal to the catheter tip or in a handle. The measurement may be correlated to flow through the tubular lumen and/or along the helix. In other terms, the measurement serves as a basis for the determination of the condition of within the tubular lumen.

While it may be desirable to monitor the flow of bodily bluid and/or bodily tissue through a catheter assembly having a rotatable helix, in some embodiments, the rotatable helix may be optional and may not be provided. However, monitoring of fluid and/or bodily tissue flow through the catheter assembly may still be desirable.

In embodiments, detecting/monitoring/measuring/determining the condition of the flow of bodily fluid and/or tissue through the tubular lumen and/or along the helix may provide a basis for controlling the catheter assembly. In particular, operation of the rotatable helix, in embodiments including a rotatable helix, may be based on the condition detected by the at least one sensor during the procedure.

Blood flow through the tubular lumen and/or along the helix may aid in cooling (a portion of) the catheter assembly, in particular the rotatable helix and the tubing during the procedure. If the catheter assembly is not cooled, the temperature may rise, and may even lead to melting of the catheter tube or breakage of entities of the catheter. Secondly, a too low static pressure in the vessel may occur as the flow of blood is stopped and a vacuum is being created in the vessel due to the continued suction applied to the vessel. This can lead to unwanted perforation of the vessel.

By way of the sensor, a more objective detection and a faster avoidance of undesirable high temperature conditions. are possible. Further, the sensor allows a user to detect and react faster to unfavourable conditions.

In some embodiments, based on feedback from the sensor and a determination of the detected condition, a catheter assembly may be configured to automatically stop and/or adjust if a predetermined condition is met (or not met) where there is an indication of sufficient or insufficient flow of bodily fluid and/or tissue. In some embodiments, feedback may be used to increase aspiration if the condition indicates that higher flow rates are feasible, such as without substantially increasing risk of undesirable temperature conditions.

The catheter assembly of the present disclosure may be specifically advantageous for procedures on small vessels, such as for vessels “below the knee”, carotid and/or coronary vessels. Generally, the catheter assembly may be beneficial for applications in which an insufficient flow is likely to occur, since the vessel is partly or fully occluded. For example, in small vessels and/or challenging occlusions, the risk for an occlusion may be increased.

As such, the catheter assemblies according to the present disclosure may allow for improved operation of the device.

The condition as detected may relate to an indirect measurement or indication of the flow of bodily fluid and/or issue. In this regard, the condition as measured/detected may allow to derive the flow rate, but does not need to be a (direct) measurement of the flow rate. Hence, the sensor may be used for an indirect detection/measurement/determination/monitoring of the flow through the catheter.

Generally, the bodily fluid and tissue may be blood with removed clot and/or calcifications.

Detecting the condition means measuring and/or determining the condition and/or monitoring the condition, with a sensor. Specifically, the condition may be monitored over a specific time, such as the entire procedure.

Optionally, the condition to be detected may be the temperature. Temperature sensors may be thermocouples or resistor-based sensors. Other conditions to be detected may include pressure or flow rate, for example, outside the catheter or inside the catheter lumen. Accordingly, the sensor may be a pressure sensor or a flow sensor. As a pressure sensor, strain gauges, piezoelectric, or capacitance-based sensors may be used. As a flow sensor velocity, volumetric, mass sensors, as well as inferential flow sensors are conceivable.

The present disclosure may include:

In another aspect, a catheter assembly for thrombectomy or atherectomy procedures, wherein the catheter assembly comprises:
a catheter having a tubing comprising a tubular lumen extending in a length direction, and a distal end,
a rotatable helix at least partially received in the tubular lumen and rotatable relative to the tubing,
at least one sensor, optionally comprised by the catheter, for detecting a temperature during the procedure.

Optionally, the sensor is located at a position along the catheter such that the sensor is received within the subject during a procedure. For example, the sensor may be located at a distal part of the catheter.

In such embodiment, the sensor is placed distal enough so as to ensure that the sensor is inside the subject during the procedure. This will reduce any uncertainty or influence due to the environment or ambient circumstances. Specifically, if the condition is measured within the subject, it is understood that the measurement may directly identify the condition (not only indirectly indicate the condition)

However, a sensor may be provided elsewhere, such as in the catheter assembly, such as in a handle of the catheter assembly. Hence, in some embodiments, the sensor is not necessarily in the catheter of the catheter assembly.

Optionally, the tubing or catheter comprises a tubular wall, and the sensor is embedded within the wall of tubing. Embedding the sensor in the tubing allows for reliable measurement and detection, as the sensor is not in immediate contact with the body, in particular the blood or the vessel wall. In embodiments, the sensor is in direct contact with the wall of the tubing. For example, the sensor may be encapsulated by the wall.

One or more wires may run along the wall of the tubing in the length direction from the at least one sensor. The wires may be embedded within the wall. These wires serve for a connection between the sensor and an entity outside the tubing/catheter. If the wires are embedded within the wall, the diameter of the catheter tube may not need to be increased, so that the profile may be kept small.

Optionally, the catheter assembly comprises a control unit for controlling the catheter assembly, in particular rotation of the helix, based on the condition detected. Hence, the at least one sensor may be used to provide the detected condition (a signal representative of the condition) to a control unit. Such control unit may use this signal as information for controlling the catheter assembly. Specifically, the control of the catheter assembly relates to the rotation of the helix, such as the angular velocity, which may be determined by the power supplied by a motor for rotating the helix. If the condition as detected represents or indicates a flow through the tubular lumen and/or along the helix, which flow is too low, the control unit may stop rotation of the helix or may reduce the rotational speed of the helix, and may issue an alert for warning the practitioner. If the condition as detected represents or indicates a flow which high enough, the control unit may initiate a faster rotation of the helix, which increases the aspiration.

Generally, the condition which indicates the flow of the bodily fluid and/or tissue through the tubular lumen and/or along the helix can be used to control the catheter assembly and/or to inform the user about the condition indicative of flow of bodily fluid and/or tissue through the tubular lumen and/or along the helix. For example, if the detected condition indicates a flow outside of a desired range, a warning may be issued to the practitioner, such as an acoustic signal or tactile signal such as vibration. A speaker or a vibrator may be used, respectively. The practitioner may need to stop or at least interrupt the treatment in response to the warning.

Optionally, the control unit comprises a closed loop control for controlling the rotation of the helix based on the condition as detected. If a closed loop control is applied, there may be no need for the practitioner to take any measure if the flow of bodily fluid and/or tissue is outside a desired range. The closed loop control may allow for automatic adjustment so as to lead to a change of the condition such that a condition which meets a desired range is met.

Generally, the temperature of the catheter may increase because of the rotation of the helix, as the helix rotates relative to the tubing, which entails frictional forces between the helix and the tubing. Any rotating parts (such as the helix, and the rotatable part of the head, for example) may cause friction relative to stationary entities (such as the catheter tube, and the stationary part of the head, for example). The rotational speed of the helix is a decisive factor in connection with friction, as the helix rotates at a fast speed, for example 40,000 to 60,000 rotations per minute.

In embodiments, the catheter further comprises a head distal to the tubing. The head is provided at the distal end of the catheter. Optionally, the head comprises a stationary part, which is stationary relative to the catheter tube. In this case, friction between the rotating helix and the stationary part, at the distal abutment, may increase the temperature of the catheter. Further optionally, the head may additionally comprise a rotatable part, the rotatable part rotatable relative to the tubing and the stationary part which is stationary relative to the tubing. The rotatable part is connected to the helix such that rotation of the helix imparts rotation to the rotatable part. In this case, friction may occur between the stationary part and the rotatable part.

Optionally, the rotatable part may comprise the at least one sensor. The sensor may be provided within the rotatable part.

The embodiments above are described in connection with one sensor. However, a plurality of sensors may be provided. For example, one sensor may be provided at the tubing of the catheter, and another sensor may be provided at the rotatable part of the head of the catheter. Other combinations are conceivable, such as a plurality of sensors at the catheter, for example on the tubing and on the head.

Multiple sensors in the tubing may be arranged, optionally equidistantly. The same distance may refer to radial and/or the length direction of the tubing.

In embodiments, the catheter assembly further comprises a guidewire to be at least partially received within the lumen and to extend at least partially along the rotatable helix. In particular, the guidewire may extend along the length direction within the rotatable helix. Put differently, the guidewire may at least partially be surrounded by the rotatable helix. In this case, the rotatable helix has a central and elongated cavity which extends along the length of the helix, in the length direction, in which the guidewire can be received.

If a guidewire is used together with such helix, the helix rotates relative to the guidewire. This may lead to friction between the guidewire and the helix, thereby generating heat within the device and/or vessel.

Optionally, the sensor or a plurality of sensors may be provided at the distal end of the catheter, optionally up to about 0.2 m to 0.6 m proximal to the distal end of the catheter. In other words, a plurality of sensors may be provided at the catheter's distal end, optionally distributed along the catheter's distal end, within a region extending from the distal end to about 0.2 m proximal to the distal end; or a corresponding region of about 0.3, 0.4, 0.5 and 0.6 m. This should ensure that the sensor is received within the subject during the treatment.

The sensor may be a temperature sensor. In this case, the sensor may be a thermocouple, for measuring the temperature of the catheter. Two wires of the thermocouple may connect the sensor to another entity. Such wires may extend along the length direction and within the wall of the tubing. For example, the two wires may extend on opposite sides of the circumference of the wall, meaning that between the wires, the tubular lumen of the tubing is positioned.

Optionally, the tubing is made of polymer. Additionally or alternatively, a braided tubing may be used. This reinforcement of the tubing allows for reliable and convenient realization of the various embodiments of the present disclosure.

Embodiments of the present disclosure may include a control unit for controlling a catheter assembly in thrombectomy or atherectomy procedures. For example, embodiments of the catheter assembly may be controlled by the control unit. The control unit may comprise a closed loop control for controlling a drive unit for rotating the helix, wherein the power with which the helix is rotated and/or the angular speed/velocity at which the helix is rotated, is based on the condition detected by the at least one sensor.

Optionally, the closed loop control is for stopping and/or adjusting (e.g., reducing) the power supplied to the drive unit and/or for stopping and/or adjusting (e.g., reducing) the rotation of the helix, if the condition as detected does not meet a predetermined setting. This may ensure that a the system may automatically respond the changes in the condition without input by a user, thereby preventing undesirable conditions within the vessel and/or the device. That is, the closed loop control allows for automatic monitoring and stopping/adjusting. An automatic stop or adjustment may be faster than a stop initiated by the user. Hence, the automatic control may be faster to react to unfavourable incidences, thereby further reducing safety risk to the subject.

Optionally, the condition as detected is indicative of flow of bodily fluid and/or tissue through the tubular lumen and/or along the helix, wherein further optionally the condition which is detected is the temperature.

Optionally, the control is continuously applicable during a procedure.

The present disclosure further includes embodiments directed to a handle for thrombectomy and atherectomy procedures. In particular, the handle may be re-usable, whilst the catheter assembly may be a disposable and a single use assembly.

The handle may be for one or more embodiments of the catheter assembly described herein and may comprise a housing accommodating a motor for rotating the helix of the catheter assembly, an (signal) inlet or receiver for receiving a signal indicative of the condition as detected by the at least one sensor, and an (signal) outlet or transmitter for transmitting to a control unit, optionally the control unit of the present disclosure, a signal indicative of the condition as detected by the at least one sensor. The signal received by the inlet and the signal transmitted by the outlet may be the same, but may alternatively be different.

As such, a handle provides for the control as suggested and for transmitting the signal which represents the condition as detected to a control unit.

Optionally, the inlet is an electrical inlet for connecting to one or more wires of the catheter (directly or indirectly), the sensor is connected to the one or more wires for connection to the control unit. Optionally or additionally, the outlet is an electrical outlet for connecting to a control unit for driving the motor.

### BRIEF DESCRIPTION OF THE DRAWINGS

FIG. 1a and 1b schematically depict cross-sectional views of a catheter perpendicular to and along the length direction, respectively, according to one or more embodiments shown and described herein.

FIGS. 2a and 2b schematically depicts a distal part of a catheter and a cross-sectional view of a part of a catheter along the length direction, respectively, according to one or more embodiments shown and described herein.

FIG. 3a to 3c show various embodiments with respect to the position of the sensor in the radial direction.

FIG. 4 shows a distal part of the tubing and the position of the sensor in the length direction, according to one or more embodiments shown and described herein.

FIGS. 5a and 5b show different embodiments with respect to the angular position of multiple sensors.

FIG. 6 shows a system including a catheter assembly, according to one or more embodiments shown and described herein.

### DETAILED DESCRIPTION

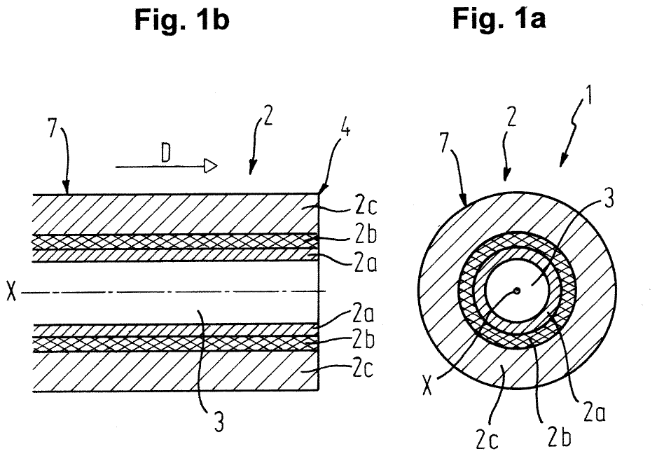

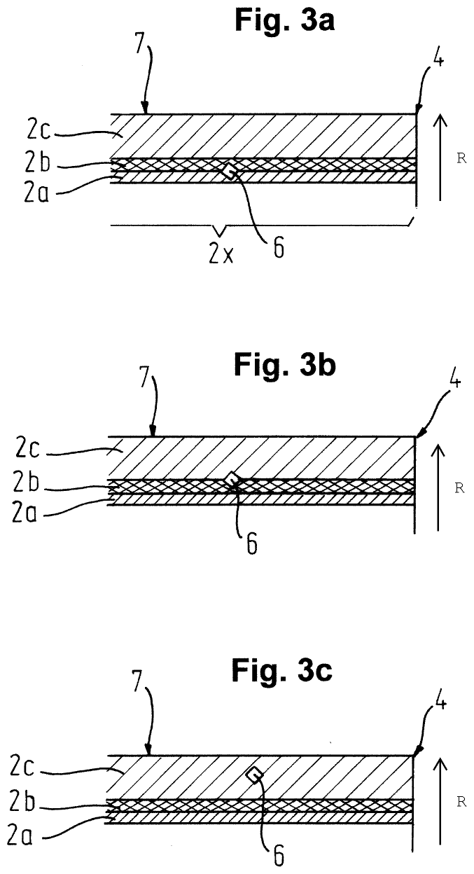

The present disclosure is directed to a catheter assembly for thrombectomy or atherectomy procedures. FIGS. 1a and 1b show that the catheter assembly including a catheter 1 having a tubing 2 defining a tubular lumen 3 extending in the length direction D. The tubular lumen 3 is defined by a tubular wall 7. The tubing 2 comprises the tubing or tubular wall 7 and a distal part 2x (cf. FIG. 3a). The distal part 2x may be at the distal end 4 of the catheter 1/of the tubing 2, but the distal end 4 may be further distal than the distal part 2x of the tubing, see FIG. 3a.

FIG. 1a is a cross-section of the catheter tubing 2 taken along the direction perpendicular to the length direction D, while the cross-section of FIG. 1b is taken along the length direction D. In embodiments, the tubing wall 7 may be formed of or have at least three layers. The layers are, in this embodiment as follows, from inward to outward: a (inner) liner 2a, a (middle) braiding 2b, and an (outer) jacket 2c.

These layers of the tubing 2 or wall 7 are concentric and centred relative to the central axis X of the catheter tubing 2. The material of the liner 2a may be a polymer with a thickness, optionally between 20 μm to 100 μm thick, the braiding 2b may be stainless steel, and/or the jacket 2c may be made of polymeric material, with a thickness optionally ranging from 100 μm to 500 μm. In another embodiment, the tubing 2 may be made of a polymer and may, optionally, be braided.

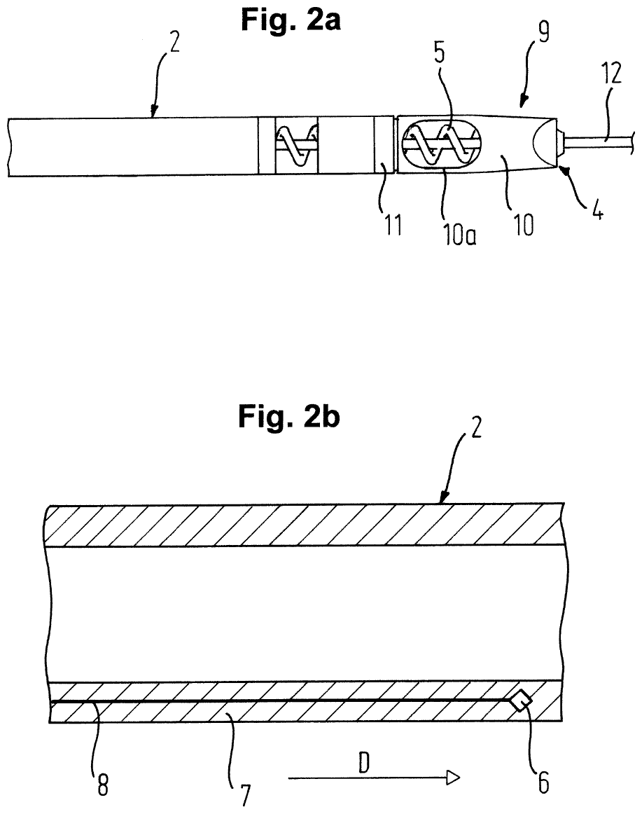

At the distal end 4 of the catheter, a head 9 may be provided, see FIG. 2a. The head 9 is provided distal to the tubing 2, and is, hence, provided at the distal end/part 4 of the catheter tubing 2. The head 9 may include a rotatable part 10 and a stationary part 11. The rotatable part 10 is rotatable relative to the tubing 2, and the stationary part 11 is stationary relative to the tubing 2. The rotatable part 10 may cover or encapsulate the stationary part 11. Both parts 10, 11 have or define an opening for aspiration of clot/calcifications etc. In FIG. 2, an embodiment of an opening 10a in the rotatable part 10 is shown, the opening in the stationary part 11 “falling within” the opening 10a, i.e. the openings are at least partially congruent. Put differently, the opening of the stationary part 11 is aligned with and has the same size as the opening 10a. The rotatable part 10 may be connected to a helix 5, so that rotation of the helix 5 imparts rotation to the rotatable part 10. Hence, the rotatable part 10 may rotate together with the helix 5. A guidewire 12 extends through the catheter 1 in the length direction D.

The rotatable helix 5, is at least partially received in the tubular lumen 3, as indicated in FIG. 2a. The helix 5 is rotatable relative to the tubing 2. Specifically, the rotatable helix 5 is rotatable around the axis X extending in the length direction. The tubing 2, in particular the tubular lumen 3, and the rotatable helix 5 have the common central axis X and are coaxial.

The catheter assembly comprises the guidewire 12, which is at least partially received within the lumen 3, see FIG. 2a. The rotatable helix 5 extends at least partially around the guidewire 12. The helix 5 may surround the guidewire 12, at least partially in the length direction. More specifically, the helix 5 may form an elongate cavity in the centre of the helix 5, and the guidewire 12 may be received within this elongate cavity.

According one or more embodiments, the catheter 1 includes at least one sensor 6. The sensor 6 is for detecting a condition during the procedure. Put differently, the sensor 6 may measure or determine or monitor a condition during the procedure.

The sensor 6 is embedded within the wall 7 of the tubing 2, see FIG. 2b. The sensor may, alternatively, or additionally, be provided at the head 9, in particular the rotatable part 10 (not shown in the drawings).

One or more wires 8 may run along the wall 7 of the tubing 2 in the length direction D from the at least one sensor 6, as shown in FIG. 2b.

Various “depth” positions of the sensor 6 are shown in FIG. 3a to 3c. These views on the position of the sensor 6 refers to the depth in the wall 7. For example, in the embodiment of FIG. 3a, the sensor 6 is located (sandwiched) between the inner layer 2a (a liner) and the middle layer 2b (a braiding) of the tubing 2. The sensor 6 is, accordingly, located at the boundary between the inner layer 2a and the middle layer 2b. Alternatively, in the embodiment of FIG. 3b, the sensor 6 is located (sandwiched) between the middle layer 2b and the outer layer 2c (a jacket) of the tubing 2. The sensor 6 is, accordingly, located at the boundary between the middle layer 2b and the outer layer 2c.

Alternatively, in the embodiment of FIG. 3c, the sensor 6 is embedded in the outer layer 2c. In embodiments with more than one sensor 6, the multiple sensors 6 may be at different positions shown in FIG. 3a to 3c.

The condition the sensor 6 detects is indicative of the flow of bodily fluid and/or tissue through the tubular lumen 3 and/or along the helix 5. The flow is of interest, as an occlusion or the presence of the catheter may lead to reduced or no flow of blood. Specifically, the sensor 6 may be a temperature sensor, which means that the condition to be detected is the temperature. In the embodiments shown, the temperature sensor is a thermocouple. Alternative temperature sensors may be resistor-based.

The condition which indicates the flow of body fluid may be the temperature of the catheter. As explained above, if the flow of body fluid is too low, the temperature of the catheter 1 may rise. The sensor 6 is located at a position which is received within the subject during a procedure. Specifically, this position is at a distal part of the catheter 1. The sensor 6 may be provided at a distal end 4 of the catheter, specifically the head 9 of the catheter, and/or a distal part 2x of the tubing.

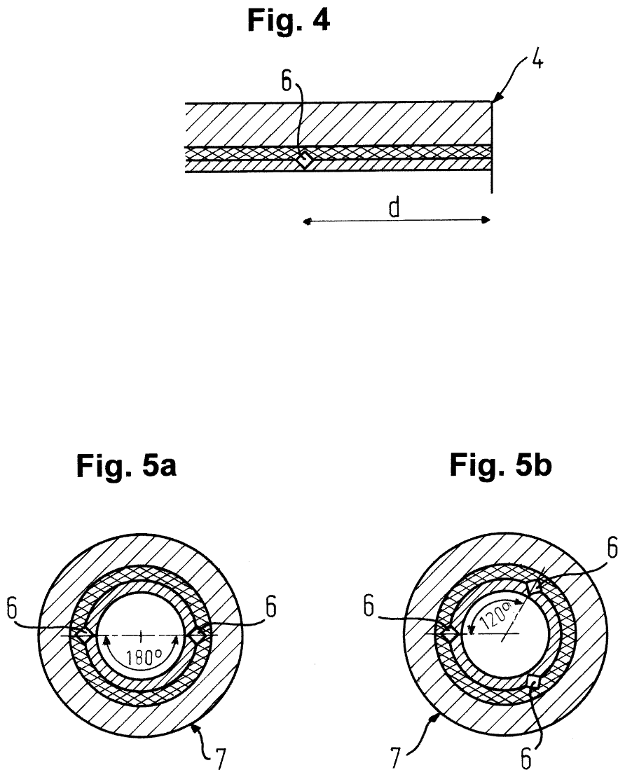

In some embodiments, the sensor may be provided at most 0.6 m proximal to the distal end 4 of the catheter. For example, the sensor may be provided within a range d of 0.1 to 1.0 m proximal to the distal end 2x of the tubing 2, or proximal to the distal end 4 of the catheter as indicated in FIG. 4. If multiple sensors 6 are provided, a distance between one sensor to the subsequent sensor 6, seen in the length direction D, may be between 0.02 to 0.10 m. Multiple sensors 6 may all be at the same distance relative to each other, i.e. they may be arranged equidistantly in the length direction D.

If multiple sensors 6 are provided, they may be arranged to have equal (angular) distances relative to each other in a cross-section perpendicular to the length direction D, e. g. in a radial aspect. When viewed from the center line X, the angles of lines extending from the center (representing the center line X) to a respective sensor in a cross-sectional view are equal. For example, in case of two sensors 6, the sensors 6 may be opposite to each other, i.e. at an angle of 180 degree relative to each other, see FIG. 5a. If three Sensors are used, they may be distributed over angles of 120 degree, see FIG. 5b. In general, if n sensors 6 are provided, the angle between the sensors is 360/n degree, i.e. equal.

Each of the sensors 6 may be at the same “depth” with respect to the tube wall 7 or may be at different depths, as indicated in FIG. 3a to 3c. The depth refers to the radial direction R.

For example, an embodiment may have three sensors 6, which have the same distance relative to each other in the length direction D and are distributed as shown in FIG. 5b around the center line X. Such sensors 6 may each be embedded in the tubing 2, more specifically between the inner layer 2a and the middle layer 2b.

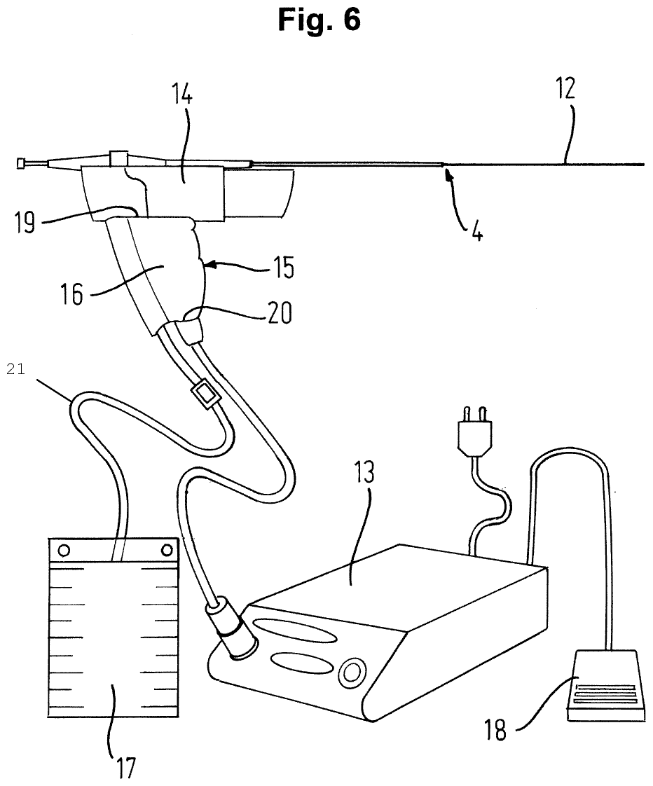

FIG. 6 shows a thrombectomy or atherectomy system. A control unit 13 for controlling the catheter assembly is provided, see FIG. 6. The control of the catheter assembly may relate to the rotation of the helix 5, based on the condition detected. The control unit 13 may comprise a closed loop control for controlling the rotation of the helix 5 based on the condition as detected. Put differently, the control unit 13 for controlling the catheter assembly may comprise a closed loop control for controlling a motor 14 for rotating the helix 5, wherein the power with which and/or the angular velocity at which the helix 5 is rotated is based on the condition detected by the at least one sensor 6. The motor 14 is in a handle 15 which the practitioner holds in the hand during the procedure.

The control unit 13 may comprise a PLC (programmable logic controller).

As an example, based on the input parameter (such as temperature or flow rate or pressure), a corrective action is decided using the control software. There may be a PLC (programmable logic controller) relating the input level to the corrective actions. No correlation table, e.g. correlating temperature to flow rate, may be needed, because they may be assessed independently for the corrective action.

The closed loop control is for stopping and/or adjusting (changing, in particular reducing) the power supplied to the drive unit 14 and/or the rotation of the helix 5, if the condition detected does not meet a predetermined setting.

The condition as detected is indicative of flow of bodily fluid and/or tissue through the tubular lumen 3 and/or along the helix 5. The control may be continuously applied during the procedure, i.e. during the entire procedure.

For example, conditions that may require the power to be stopped or reduced may be as follows: When looking at the flow rates, a flow drop below 50% of the nominal flow rate may trigger an alert to the practitioner. For an exemplary catheter of 6Fr, 8Fr, and 10Fr with, respectively, nominal flow rates of 45 ml/min, 75 ml/min, and 130 ml/min, a flow rate drop below 22 ml/min, 37 ml/min and 75 ml/min, respectively, may be considered as the signal threshold. (“Fr” denotes the French scale/gauge system and refers to the diameter of the catheter tube. The remaining parts, such as the head, are sized accordingly.) In some embodiments, a flow drop below 5, 10 or 20% of the nominal flow rate may be a threshold for reducing or stopping use, though other thresholds are contemplated and possible.

When looking at the temperature, in some examples, a temperature rise to above about 25, 30 or 35% of the melting point of the polymers used in the catheter may trigger an alert to the practitioner. Whereas a temperature rise to about 50% of the melting point, may be a threshold for reducing or stopping use. For example, if the polymer used in the catheter has a melting point of 180 degrees Celsius, a temperature rise detection of above 54 degrees Celsius may trigger an alert, while a temperature rise to above 90 degrees Celsius may be the trigger to reduce or stop the use.

In embodiments, a re-useable handle 15 for thrombectomy or atherectomy procedures 15 may be used. The handle 15 has a housing 16 accommodating the motor 14 for rotating the helix 5 of the catheter assembly. The handle 15 may further include a signal inlet 19 for receiving a signal indicative of the condition as detected by the at least one sensor 6. The handle 15 may further include a signal outlet 20 for transmitting a signal indicative of the condition as detected by the sensor 6 to the control unit 13. The inlet 19 is an electrical inlet for connection to one or more wires 8 of the catheter, wherein the sensor 6 is connected to the one or more wires 8 for connecting to the control unit 13. The outlet 20 is an electrical outlet for connection to the control unit 13 for driving the motor 14.

FIG. 6 also shows a collection bag 17 for collecting the removed tissue/bodily fluid and an optional foot switch 18 for controlling the system.

It is, in an alternative embodiment, conceivable that the sensor 6 is provided at an outlet tube 21 at the handle 15. (Additionally, a sensor 6 may be included in the catheter; however, this is not necessary.) The temperature of the clot/blood as removed from the subject after having exited the subject still allows to derive the temperature of the clot/blood inside the catheter (as it has not yet cooled completely when inside the handle or entering the collection bag 17). For example, an estimation of the typical temperature decrease can be made and be applied to derive the temperature within the subject/catheter, based on the temperature of the clot/blood measured in the handle. Therefore, a sensor at the handle 15 and/or at the outlet tube 21 may be contemplated for temperature sensor. However, for a sensor in the handle, a flow sensor may be preferable. Examples for a flow sensor are velocity, volumetric, mass, or inferential flow sensors.

During a procedure or treatment, the following scenario is possible with a catheter and control unit of the disclosure:
After (optional) priming, the catheter assembly is inserted into the subject. During subsequent treatment, if the flow of bodily fluid is reduced below a threshold, the control unit may immediately detect that the flow rate is too low. If the temperature is reduced below a threshold, the control unit may immediately detect that the temperature is too high. The catheter assembly may be automatically stopped or the rotational speed may be adjusted by the control unit, in particular to avoid (further) damage. The treatment may be continued once the source/origin of the reduced flow rate has been solved. It may be that one or more of the following “corrective” steps are needed before the treatment can be continued: Removal and flushing of catheter, (manual) reversal of rotation direction of helix (by the user), or replacement of parts.

### LIST OF REFERENCE SIGNS

1 catheter
2 tubing
2a inner layer (liner)
2b middle layer (braiding)
2c outer layer (jacket)
2x distal part of tubing
3 tubular lumen
4 distal end of catheter
5 rotatable helix
6 sensor
7 tubing wall
8 wire(s)
9 head
10 rotatable part
10a opening
11 stationary part
12 guidewire
13 control unit
14 motor
15 handle
16 housing
17 collection bag
18 foot switch
19 electric signal inlet
20 electric signal outlet
21 outlet tube
D length direction
R Radial direction
X central axis
d distance of sensor relative to distal end of catheter

1. A Catheter assembly for thrombectomy or atherectomy procedures, wherein the catheter assembly comprises:
a catheter having a tubing defining a tubular lumen extending in a length direction (D), and a distal end,
a rotatable helix at least partially received in the tubular lumen and rotatable relative to the tubing,
at least one sensor, optionally comprised by the catheter, for detecting a condition during a procedure, wherein the condition is indicative of flow of bodily fluid and/or tissue through the tubular lumen and/or along the helix.

2. The Catheter assembly of claim 1, wherein the at least one sensor comprises a plurality of sensors spaced equidistantly as to a radial and/or length position.

3. The Catheter assembly of claim 1, claims, wherein the tubing comprises a tubular wall, wherein the at least one sensor is at least partially embedded within the tubular wall of the tubing and/or wherein one or more wires run along the tubular wall of the tubing in the length direction (D) from the at least one sensor.

4. The Catheter assembly of claim 1, claims, further comprising a control unit communicatively coupled to the at least one sensor and operable to control rotation of the helix, based on the condition detected.

5. The Catheter assembly of claim 4, wherein control unit comprises a closed loop control for controlling the rotation of the helix based on the condition as detected.

6. The Catheter assembly of claim 1, the catheter further comprising a head distal to the tubing, the head comprising a stationary part, and a rotatable part, the rotatable part rotatable relative to the tubing and the stationary part stationary relative to the tubing, the rotatable part connected to the helix such that rotation of the helix imparts rotation to the rotatable part.

7. The Catheter assembly of claim 6, wherein the at least one sensor is provided at the head, the rotatable part within the rotatable part.

8. The Catheter assembly of claim 1, the catheter assembly further comprising a guidewire to be at least partially received within the lumen and to extend at least partially along the rotatable helix, to be at least partially surrounded by the rotatable helix, at least partially in the length direction (D).

9. The Catheter assembly of claim 1, wherein the sensor is provided at the distal end of the catheter, at the head and/or a distal part (2x) of the tubing, about 0.2 to 0.6 m proximal to the distal end of the catheter.

10. The Catheter assembly of claim 1, wherein the sensor is a thermocouple, for measuring the temperature of the catheter.

11. The Catheter assembly of claim 1, wherein the tubing is made of polymer and/or is braided.

12. A Control unit for controlling a catheter assembly in thrombectomy or atherectomy procedures, wherein the catheter assembly, comprises:
a catheter having a tubing defining a tubular lumen extending in a length direction (D), and a distal end,
a rotatable helix at least partially received in the tubular lumen and rotatable relative to the tubing,
at least one sensor for detecting a condition of the catheter during the procedure, wherein the condition is indicative of flow of bodily fluid and/or tissue through the tubular lumen and/or along the helix,
wherein the control unit comprises a closed loop control operable to control a drive unit configured to rotate the helix, wherein the power with which and/or the angular velocity at which the helix is rotated, is based on the condition detected by the at least one sensor.

13. The Control unit of claim 12, wherein the closed loop control is configured to stop the power supplied to the drive unit and/or the rotation of the helix, in response to the condition detected being outside of a predetermined setting.

14. The Control unit of claim 12, wherein the condition detected is indicative of flow of bodily fluid and/or tissue through the tubular lumen and/or along the helix, wherein the condition which is detected is the temperature.

15. The Control unit of claim 12, wherein the control unit is configured to monitor the condition throughout the procedure.

16. A Handle for thrombectomy or atherectomy procedures, for a catheter assembly comprising
a catheter having a tubing defining a tubular lumen extending in a length direction (D), and a distal end,
a rotatable helix at least partially received in the tubular lumen and rotatable relative to the tubing,
at least one sensor comprised by the catheter for detecting a condition of the catheter during the procedure, wherein the condition is indicative of flow of bodily fluid and/or tissue through the tubular lumen and/or along the helix,
handle comprising
a housing accommodating a motor for rotating the helix of the catheter assembly,
a signal inlet for receiving a signal indicative of the condition as detected by the at least one sensor,
a signal outlet for transmitting to a control unit control signal indicative of the condition as detected by the at least one sensor.

17. The Handle of claim 16, wherein the signal inlet is an electrical inlet for connection to one or more wires of the catheter, the sensor connected to the one or more wires, and/or the outlet is an electrical outlet for connection to a control unit for driving the motor.

18. Use of a catheter assembly for thrombectomy or atherectomy procedures, wherein the catheter assembly comprises:
a catheter having a tubing defining a tubular lumen extending in a length direction (D), and a distal end,
a rotatable helix at least partially received in the tubular lumen and rotatable relative to the tubing,
at least one sensor, comprised by the catheter, for detecting a condition during a procedure,
wherein the use comprises identifying a condition indicative of flow of bodily fluid and/or tissue through the tubular lumen and/or along the helix.
